(19) United States
(12) Patent Application Publication
(10) Pub. No.: US 2018/0355405 A1
(43) Pub. Date: Dec. 13, 2018

(54) System and Method for Determining Glucose Oxidase in an Analyte

(71) Applicant: Accessible Diagnostics, LLC, Greenville, SC (US)

(72) Inventors: Rebecca B. Silveston-Keith, Columbia, SC (US); Katherine L. Hafner, Columbia, SC (US); John Riley Warner, III, Greenville, SC (US)

(73) Assignee: Accessible Diagnostics, LLC, Greenville, SC (US)

(21) Appl. No.: 16/001,910

(22) Filed: Jun. 6, 2018

Related U.S. Application Data
(60) Provisional application No. 62/517,413, filed on Jun. 9, 2017.

(51) Int. Cl. C12Q 1/54; C12Q 1/26; G01N 1/00; G01N 21/78; G06T 7/90

(52) U.S. Cl. CPC C12Q 1/54; C12Q 1/26; C12Y 101/03004; G01N 2021/7776; G01N 21/78; G06T 7/90; G01N 2001/002; G01N 1/00

(57) ABSTRACT

The present invention is a system for determining and reporting results from a test including a stick having a collection area configured to collect an analyte; a reagent for application on the analyte to provide a post reaction analyte color; a visual scale having a plurality of hues and operatively associated with the post reaction analyte color; and, computer instructions having an image capture system for capturing a digital image of the visual scale and the post reaction analyte color, determining the contrast between hues of the visual color scale, assigning a range of color to each hue, assigning a blood glucose value to each range of color, determining the range of color containing the post reaction analyte color, retrieving the blood glucose value associated with the hues of the range containing the color of the post reaction analyte, displaying the blood glucose level on the remote computer device.

### BACKGROUND OF THE INVENTION

### 1) Field of the Invention

This invention is directed to a non-invasive system and method of determining the existence or level of an analyte in a mammal's body fluid such as when analyzing a diabetic's blood for a determination of its relative glucose level.

### 2) Description of Related Art

Clinical testing of mammal's body fluids plays a significant role in the diagnosis and management for a variety of diseases. For example, blood glucose monitoring is one of the principal methods used to manage diabetes. As shown in U.S. Pat. No. 4,890,621, one process involves providing a heath care facility with a specimen of urine or blood, which is then analyzed with a strip impregnated with a reagent so that, when it reacts with the bodily fluid, it can indicate blood glucose levels. There have been improvements allowing for the monitoring of blood glucose without having to physically travel to the health care facility. U.S. Pat. No. 6,561,989 discloses a test sensor for use in the determination of the concentration of a chemical in blood, the test sensor having a flat test chamber disposed between front and rear panels having an inlet where the test chamber is used to collect a sample of blood through the inlet. A lance is included to puncture skin allowing blood to flow into the test chamber. U.S. Pat. No. 5,426,032 is directed to using a apparatus using an enzyme substrate which results in the production of hydrogen peroxide as an enzyme product that can be read by an optical device without separating whole blood. While the portable nature of these devices allows users to conveniently test blood glucose levels remote from the health care provider, they pose some health risks and can be painful. For example, the Centers for Disease Control and Prevention (CDC) have become increasingly concerned about the risks for transmitting hepatitis B virus and other infectious diseases during assisted blood glucose monitoring due the requirement that the skin is pricked.

In order to reduce or eliminate the disadvantage and risks with these invasive testing methods, attempts have been made to create non-invasive testing such as disclosed in U.S. Pat. No. 6,675,030. This patent discloses a system that creates a modeling equation for predicting blood glucose values that is generated as a function of non-invasive spectral scans of a body part and an analysis of blood samples from the patient. However, this method relies upon spectrometry and while stating that it is non-invasive, discloses that the “dynamic model is accomplished by scanning the subject with a noninvasive spectroscopic blood glucose monitor and then using an invasive technique (e.g., venipuncture or a fingerstick) to obtain a constituent value to associate with the spectral data.”

As reported in 2014 by the CDC, there are estimated to be 21 million individuals with diagnosed diabetics and 8 million that are undiagnosed which collective make up about 9 percent of the United States population. However, diabetes is not limited to humans. A 2011 study of 2.1 million dogs and 450,000 cats by Banfield Pet Hospital shows increases in diabetes and other common health problems in pets. This report states that there has been a 32% increase in canine diabetics and 16% increase in feline diabetes of cases at Banfield Hospital. Further, other animals/mammals can be affected by diabetes, such as horses, where it has been referred to as “insulin resistance” or “pre-Cushing's”. Further, diabetes is a relatively common endocrinopathy in the cat as stated in United States Patent application 2005/0026826. While attempts have been made to treat the diabetes in animals such as United States Patent Application 2016/0166581, the testing methods used largely are those taken from human testing.

As reported by VCA Hospital, Inc., the following screening tests are performed when diabetes mellitus is suspected in a feline: a complete blood count (CBC), a serum biochemistry profile, and a urinalysis. These methods do not easily lend themselves to home monitoring of an animal and blood sampling and testing are the current methods of “at home” animal blood glucose monitoring such as devices described in U.S. Pat. No. 9,549,694.

It would be advantageous to have a blood glucose testing device that can be used without having the draw blood and that can be used in the “at home” environment.

Some attempts have been made to use bodily fluid that can be collected without breaking the skin such as by using salvia. For example, U.S. Pat. No. 3,947,328 discloses a method and apparatus for rapid, accurate test of concentration levels of glucose in saliva. This reference uses oxidization of the test sample under controlled conditions with an excess of oxidizing agent and measures the level of the glucose in the body fluid as redox potential in millivolts of a primary cell in which the residual oxidizing solution is the electrolyte. However, this method requires calibration and controls of the testing environment preventing it from being well suited for at home use. U.S. Pat. No. 6,102,872 is also directed to using salvia to provide glucose monitoring but is limited to a test strip for quantitating blood glucose level based on the amount of glucose detected in salvia. U.S. Pat. No. 9,244,035 is directed to glucose sensor suitable for measuring glucose levels in saliva but is limited to human salvia. This method is difficult and expensive to make and use as the fabrication of the sensor involves depositing single-walled carbon nanotubes onto the surface of a working electrode in a 3-electrode electrochemical detector and functionalizing the nanotubes by depositing layers of polymers, metallic nanoparticles, and glucose oxidase enzyme onto the nanotubes. The sensor can be used as a disposable, single-use device or as part of an analytical system, such as a microfluidics system, for the analysis of multiple analytes.

Accordingly, it is an object of the present invention to provide for a device that can test an analyte taken from a test subject without invasive drawings of fluids.

It is another object of the present invention to provide for a device that can test an analyte for blood glucose levels.

It is another object of the present invention to provide for a device that can test an analyte to test for blood glucose levels for humans and animals.

### SUMMARY OF THE INVENTION

The present invention is directed to a system for determining and reporting results from an analyte taken from a test subject comprising: a package having a stick pocket and a pot; a stick having a collection area configured to collect an analyte in a discrete range of volume; a reagent disposed in the pot where the stick with the analyte can be inserted in the pot allowing the analyte and reagent to mix to provide a post reaction analyte color; a visual scale having a plurality of hues and operatively associated with the post reaction analyte color; and, a set of computer readable instructions included on a remote computer device having an image capture system for capturing a digital image of the visual scale and the post reaction analyte color simultaneously, determining the contrast between hues of the visual color scale, assigning a range of color to each hue, assigning a discrete blood glucose value to each one or the ranges of color, determining the range of color containing the post reaction analyte color, retrieving the blood glucose value associated with the hues of the range containing the color of the post reaction analyte, displaying the blood glucose level on the remote computer device.

The system can include a reagent window defined in the package allowing the analyte color to be viewed which the stick is received in the package. The visual scale can be disposed on the package, stick, container, card or other items associated with the collection area. The collection area can include a plurality of ridges or a pad assembly having a cellulose pad for collecting analyte of a volume wherein the ratio of analyte to reagent is in the range of 1:1 to 1:15. The collection can collect reagent in a range of volume of 1 μl to 125 μl. The reagent can be disposed on the collection area.

The computer readable instructions can include instructions for retrieving the blood glucose value associated with the hues of the range containing the color of the post reaction analyte include retrieving blood glucose values from a lookup table containing the blood glucose value associated with each specific hue of the visual scale. The computer readable instructions can include instructions for modifying the lookup table according to test subject criteria. The lookup table can include blood glucose values associated with specific test subject and the computer readable instructions can include instructions for selecting the test subject type.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1A is a schematic of aspects of the invention;

FIG. 1B is a schematic of aspects of the invention;

FIG. 1C is a schematic of aspects of the invention;

FIG. 2 is a schematic of aspects of the invention;

FIG. 3A is a schematic of aspects of the invention;

FIG. 3B is a schematic of aspects of the invention;

FIG. 4 is a schematic of aspects of the invention;

FIG. 5 is a schematic of aspects of the invention;

FIG. 6 is a schematic of aspects of the invention;

FIG. 7 is a schematic of aspects of the invention;

FIG. 8 is a flowchart showing the operations of aspects of the invention;

FIG. 9A a schematic of aspects of the invention;

FIG. 9B a schematic of aspects of the invention;

FIG. 10A is a graph showing aspects of the invention;

FIG. 10B is a graph showing aspects of the invention; and,

FIG. 11 is a graph showing aspects of the invention.

### DETAILED DESCRIPTION OF A PREFERRED EMBODIMENT

The following description of the invention will be better understood by reference to the following drawings that are incorporated and made part of the written specification.

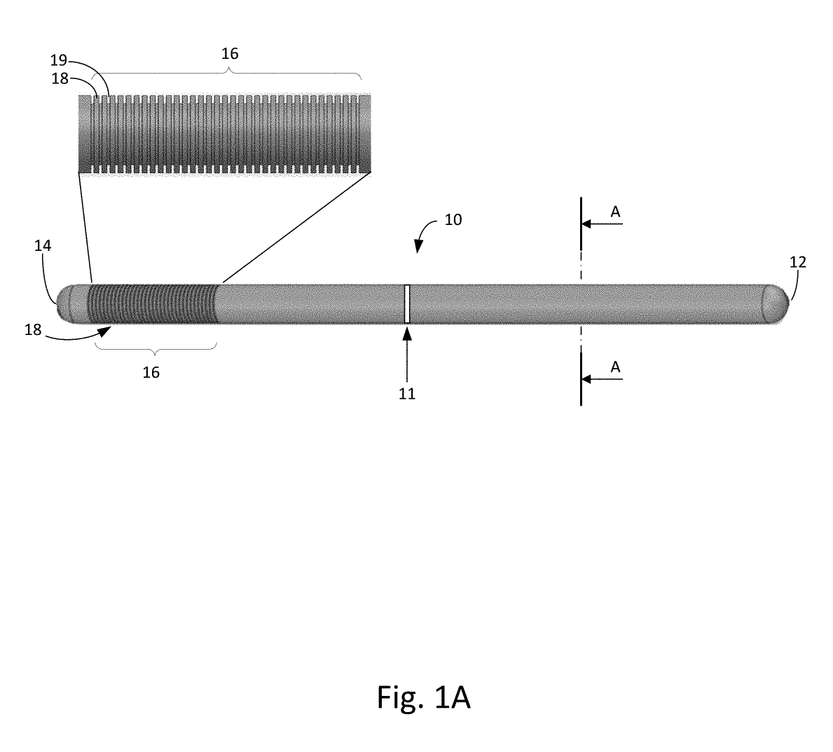

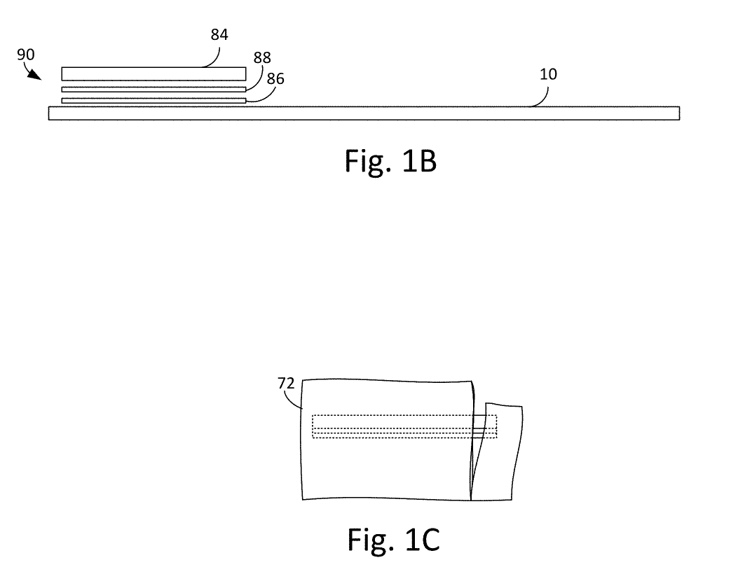

Referring to FIG. 1A, a stick 10 is shown having a proximal end 12 and a distal end 14. The distal end can include a collection area 16. The collection area can include ridges 18 that are defined in the stick and can encircle the stick on one or either end. The stick can include a cross section along A-A that is circular, oval, rectangular, square, triangular, symmetrical, asymmetrical or a combination thereof. The ends of the stick can be flat, squared, rounded or other shape. The stick can include a hinge 11 allowing the stick to be folded so that one or more ends of the stick can be better positioned in the mouth of the test subject. When folded about 180°, the stick can be used as a pincer. In one embodiment, the reagent can be embedded on or in the ridges and react when it contacts an analyte.

The ridges or valleys in the collection area can be contiguous around the stick, can be intermittent (e.g. in multiple segments with gaps between segments that can be the same size as the groove gap in one embodiment), and can be alternating or offset. The ridges need not surround the entire stick. The collection area is configured to collect an amount of fluid, such as salvia, in a discrete volume or in a range of volumes. In one embodiment, the collection area can collect a volume of salvia in a particular range so that the range of volume of salvia is known. In one embodiment, the range of salvia that is collected by the stick is in the range of 1 μl to 125 μl. In one embodiment, the ridges in the collection area can use a groove 19 having a ridge width in the range of 1 mm to 25 mm, a groove width/ridge spacing in the range of 15 mm to 50 mm, a groove depth/ridge height in the range of 15 mm to 1 cm and a draft angle in the groove in the range of 0.25° to 1.75°. The number of ridges can be in the range of 5 to 45 and corresponding to the amount of fluid/salvia that is desired to be collected. In one embodiment, the angle of the wall of ridges in relation to the long axis of the stick is in the range of 45° to 90° and in one embodiment in the range of 75° to 90°. Having a known volume of analyte (e.g. salvia), or a volume in a discrete range, allows the controlled and reproducible reaction with the reagent to determine the amount of glucose in the analyte. The collection area can capture fluid in a volume so that the volume of fluid captured compared to the volume of the reagent can be in the range of 1:1 and 1:15. The pH of a reagent in solution can be in the range of 4 to 7 and is 5 to 6 in one embodiment.

The stick can be rigid or semi-rigid. The material composition of the stick can vary and can include materials with the one or more of the following physical properties: injection moldable, 3D printed, sufficiently rigid to be bite resistant, a diameter in the range of 1/16 inch to 1 inch, sufficiently hydrophilic to allow capillary action in the ridges with fluid having viscosities in the range of 0.890 cP to 4000 cP. Exemplary materials include polymers, resins, epoxy, nylon, polyoxymethylene (POM), polyethylene terephthalate, polymethyl methacrylate polysulfone, and polyethylene glycol. The water contact angle on the material surface can be in the range of 70° to 80° wherein the contact angle can be related to the hydrophilic properties. The stick can have a length in the range of 3 inches to 12 inches.

The collection is configured to collect a discrete amount of fluid or a discrete range of fluid according to the amount of the reagent. When the ratio of the fluid to the reagent is too great, undesirable results can occur. The ratio of saliva to an enzyme reagent or other reagent can be in the range of 1:1 to 1:15, in one embodiment.

Referring to FIG. 1B, a stick 10 having the collection area that includes a pad assembly 90 at or in the collection area. The stick can be flexible in one embodiment to assist with the insertion into the subject's mouth. The pad assembly can include a pad 84 can be attached to the stick by adhesive 86 in one embodiment. The pad can be cellulose. In one embodiment, a pad can absorb about 100 uL of water. The pad can be non-toxic so that it can be placed in a subject. The cellulose pad can be white, uncolored or very lightly colored in the visible spectrum range. The cellulose pad can used alone or can be adhered to the polymer substrate 88. In one embodiment, the pad can be thermally adhered so that glues do not interfere with the chemistry on the pad. The polymer substrate can be water impervious, non-toxic and white or close to white. The polymer substrate can have a form factor that fits in a mouth. In one embodiment, the stick is about 10 cm long and 0.5 cm wide with cellulose pad being about 0.5 cm by 2.5 cm. The cellulose pad can have sufficient integrity so that it does not disintegrate in a mouth while the saliva is being collected with light rubbing (e.g. some cohesive strength when wet). In one embodiment, integrity of the pad can be such that it maintains its integrity for some pre-determined period of time such as 5 seconds. The reagent can be in the cellulose pad. The reagent can be any combination of an enzyme formulation and the pad can subsequently be used with the other ingredients in another formulation. The support material can include the printed visual scale on or near the pad including white patches for white point balancing.

Referring to FIG. 1C. the pad assembly can be stored on packet 72 and applied to the stick when needed. Multiple pads can be received in the package. In one embodiment, the pad assembly and stick can be received in the package. The stick, having the pad assembly attached, can have the pad assembly received in the package. The stick can be received on the package of have a portion of the stick extend out of the package. The reagent can be disposed on the pad or in the package so that when the analyte collected by the pad contacts the reagent, the resulting color can be analyzed as described herein. The reagent can be in a dry form or liquid.

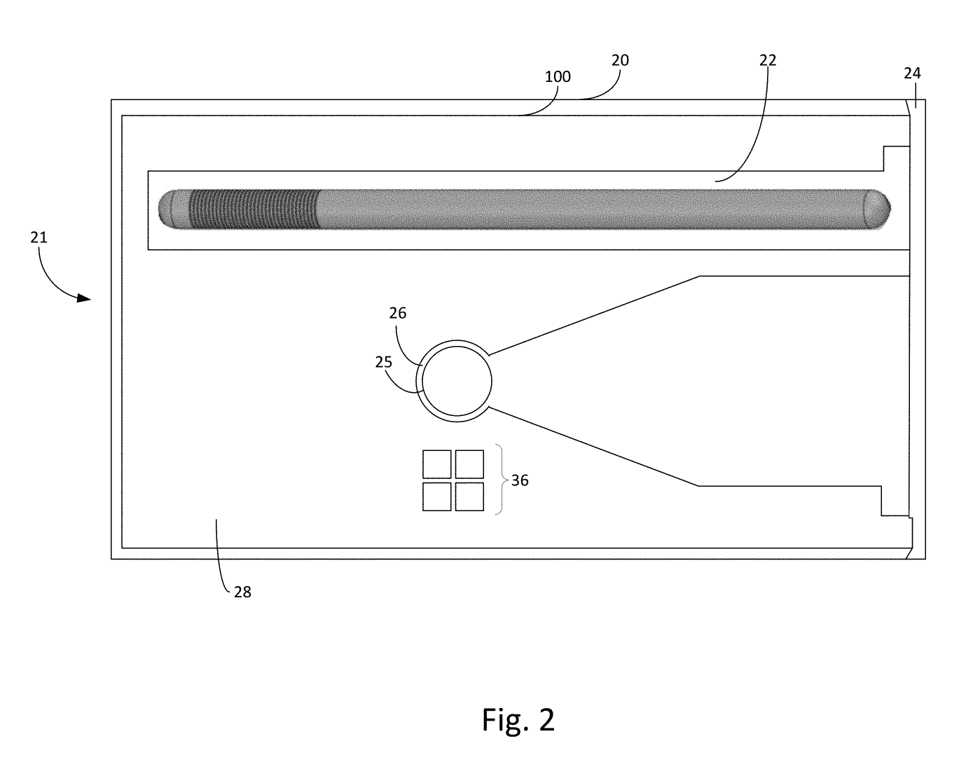

Referring to FIG. 2, the invention can include packaging 21 that can include a rear panel 100 and a front panel 20 that can be made from material such as foil. In one embodiment, the packaging can prevent gas transmission, such as oxygen, and can be non-permeable. The panels can define a stick pocket 22 that can be a space defined between the rear panel and the front panel to receive the stick. A top portion 24 can be included that can be removed from the rear and/or front panel that has a sealed position where the stick pocket is sealed with the stick received in the pocket and an opened position where the top portion can be removed providing access to the stick where the stick is removed in order to collect the analyte. The top portion can be partially removed from the rear panel and/or the front panel to provide access to the stick. A pot 26 can be defined between the rear panel and the front panel and top portion when the top portion is in the sealed position. The pot can include a reagent such as a glucose oxidase enzyme for detecting beta D glucopyranose in one embodiment. When the top portion is removed, access to the reagent is provided and the analyte, such as contained on the stick, can be placed in the pot. In one embodiment, the depth of the pot is less than the length of the stick that can prevent the stick from being fully inserted into the pot so that a portion of the stick, such as the proximal end, can be used to stir the stick in the pot to facilitate a reaction between the reagent and the analyte. The rear panel and the front panel can be attached at the edges and the stick pocket and pot defined by areas 28 where the rear panel and the front panel are attached to each other. In one embodiment, the reagent can be disposed in the pot where the stick with the analyte can be inserted. The pot can allow the analyte and reagent to provide a post-reaction analyte color viewable through a reagent window 25. An image capture system can then capture the post reaction analyte color and a visual scale 36 operatively associated with the post reaction analyte color.

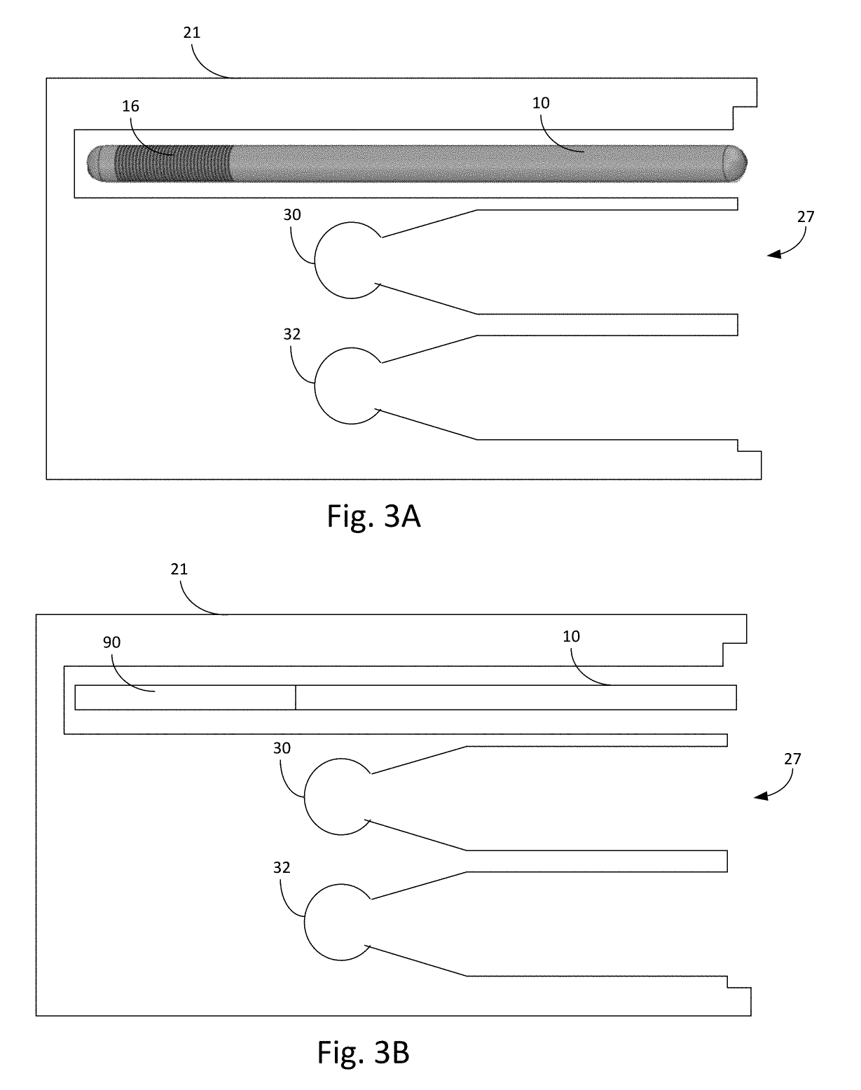

Referring to FIGS. 3A and 3B, the package 21 there can be a first pot 30 containing a reagent where the stick, with analyte, can be inserted into open area 27. A second pot 32 can then receive the stick, with analyte and the contents of the first pot, to perform a reaction. The various pots can include a dry reagent, such as an enzyme, a fluid or any combination where two materials are used to perform the reaction. In one embodiment, one pot can be for liquid on an enzyme side of the package and beta D glucose on the other pot that can be separated by a frangible seal above the enzyme solution. The user can mix the fluid laden stick in the glucose and then push it through the seal to the enzyme. The stick 10 can include a collection area having ridges as shown in FIG. 3A or having a pad or pad assembly 90 as shown in 3B.

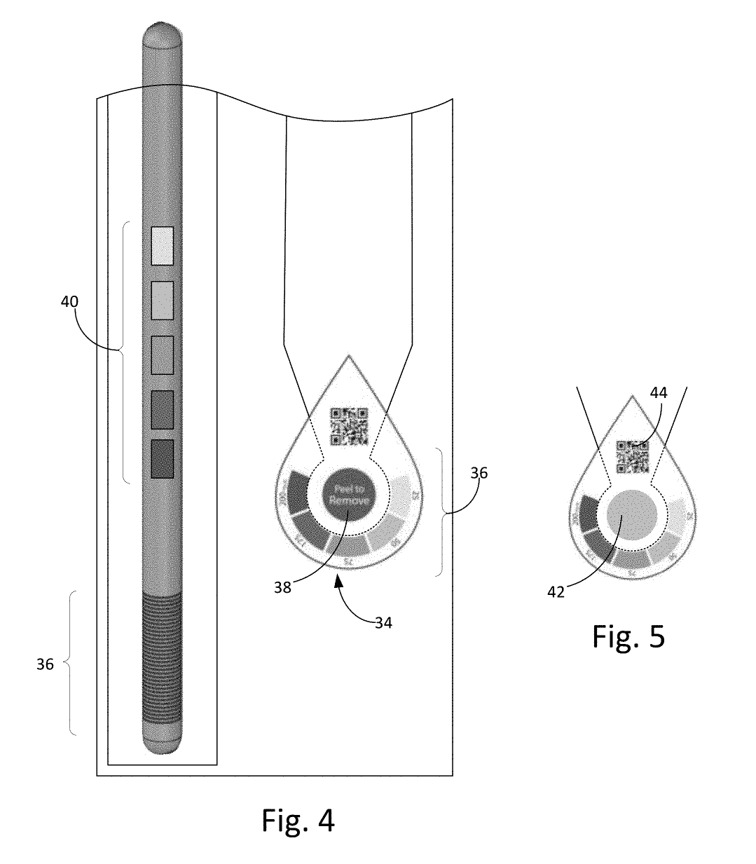

Referring to FIG. 4, the front panel is shown having an indicator area 34 that includes a visual scale 36. The visual scale can include colors in the same hue providing for a scale of blood glucose levels. Value can include 0 mg/dl to 700 mg/dl, each value or range of values associated with a color, in one embodiment. The front panel can include a window cover 38 that is removably attached to the front panel and configured to cover a transparent or semi-transparent portion of the front panel. The reagent can be disposed under the transparent or semi-transparent portion so that when the window cover is removed, the reagent or post reagent analyte color can be viewed. This cover can prevent the reagent from being exposed to UV and other light that can degrade the reagent. The visual scale can be disposed around the window or can be disposed on the stick to provide an on-stick scale 40 that can be captured with the post-reaction analyte color in area 37 on the stick. In this embodiment, the reagent can be added to the collection area and when contacting the fluid, such as salvia, uses the moisture in the fluid to facilitate a reaction. The reagent on the stick can be dry, in one embodiment. The stick pouch can be opaque to prevent or reduce degradation of the reagent due to UV or other light.

The indicator area or visual scale 34 can be used to index or align the captured image so that the location of the hues is known to the computer readable instructions. The indicator area can include an indexing member 33 allowing the image capture to determine the location, in the digital image, of the hues. The indicator area can include different hues such as 35a which can be a distinct color form the rest of the hues in the indicator area. The different hues in the indicator area can have different color so that the contrast between the colors can be determined and associated with a range of color values. Each hue can be associated with a blood glucose value. The computer readable instructions can capture the analyte color, capture the hues colors, determine the difference in color values between the hues, determine where the analyte color best falls in the range of color values between the hues, determine the associated blood glucose value associated with the analyte color when compared to the hues and display the blood glossae value to the user.

Referring to FIG. 5, when the window cover is removed, the result of the reaction between the analyte and the reagent can be seen in windows 42. An indicator 44, such as a OR code or image template that the computer readable instructions can identify or match to determine the location of the scale, can be included that can be optically or digitally scanned to asset with positioning the image capture area of the portable computer device with the image of the package capturing the post reaction analyte color and the visual scale. My matching the template, the target zone can be identified by the computer readable instructions.

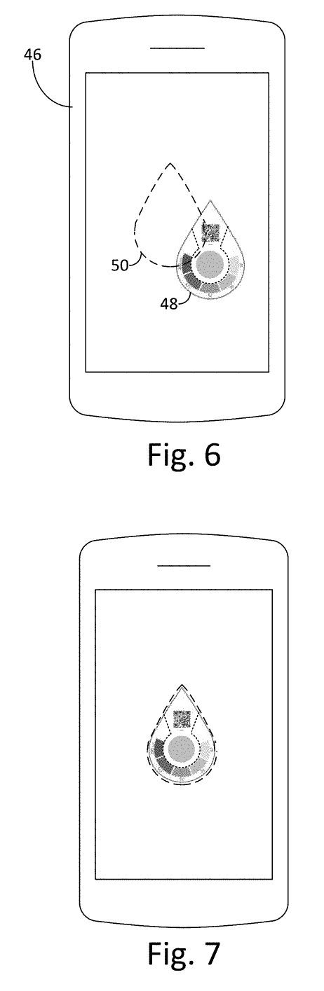

Once the stick has collected the analyte and the analyte combined with the reagent, a set of computer readable instructions can be used to interpret the results of the reaction. The computer readable instructions can be embodied on a portable computer device such as a smart phone 46 as shown in FIG. 6. The image capture system such as a camera of the smart phone can be used to capture a portion of the front panel containing the visual scale and the post reaction analyte color through window 48. The captured image can be superimposed on a placement guide 50 to allow the captured image to align with the camera of the portable computer device. The visual scale, window and placement guide are shown aligned in FIG. 7.

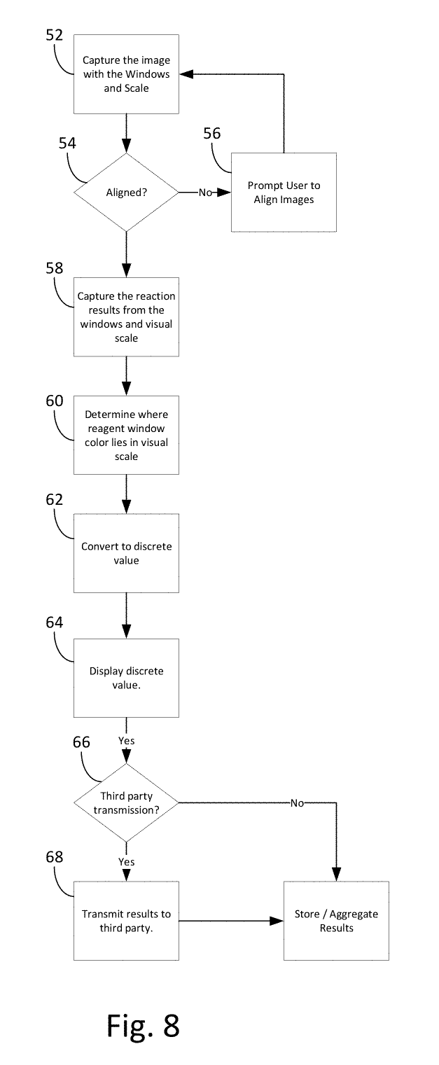

Referring to FIG. 8, the computer readable instructions can perform the function of having the remote computer device capture the visual scale and window at 52. In the capture process, the digital capture system of the remote computer device can perform a white balance to adjust to the temperature differences of the ambient light allowing for more accurate image capture in a variety of lighting conditions. A determination is made at 54 if the images are aligned. If not, the user can be prompted to align the images at 56 and repeat the process. The computer readable instructions then can capture the post-reaction analyte color in the window and the visual color scale at 58. By capturing the visual color scale with the reagent window, a determination can be made at 60 comparing the post-reaction analyte color to the visual scale to determine where in the scale the reagent window color lies. Once the position in the visual scale of the reagent window color is known, the post-reaction analyte color can be converted into a discrete value at 62. The discrete value can be a numeric value or a discrete range of values. The results can be displayed to the user at 64.

In one embodiment, the computer readable instructions can include information for third party transmission. It can be advantageous to transmit results obtained from each reading to a central location or remote location to provide for a series of readings. Such third-party can include a third-party collection source, remote application designed to collect results, a health care provider, veterinarian, remote server, or an accountability partner to help the user improve their health. If the computer readable instructions determine that transmission of the results is to occur at 66, the results are transmitted at 68. The results can be stored and aggregated at 69 both for the test subject and multiple test subjects for further analysis, trend analysis, and the like. The third party can then use the received information and transmit information or instructions to the subject or the subject's parent or owner.

In operations, the user can remove the stick from the stick pouch and use the stick to collect fluid such as salvia. The stick can be inserted into a subject's mouth to collect salvia. The stick is then exposed to the reagent such as by inserting it into the pot containing an enzyme, dry or in solution, with produces a post-reaction analyte color in a reagent window. The window cover can be removed to view the resulting post-reaction analyte color of the reaction. The reagent can also be on the stick so that the stick need not be placed on a pot. The computer readable instructions can capture the image of the visual scale and post-reaction analyte color as a digital image or real time video.

In one embodiment, the visual scale is color and is generally monochromatic such that the color is a range from a light hue to a dark hue. Having a monochromatic scale can reduce the impact of varying lighting conditions (ambient lighting). In one embodiment, the light from a remote computer device can be used and brightness controlled to assist with more consistent readings.

In one embodiment, the relationship of the stick to the pot or pouch needs not be 1:1. For example, a package may include multiple pots or pouches of reagents or enzyme solutions that are associated with one (or fewer sticks than pots) wherein the sticks can be reused. The sticks can be washed dried and reused for subsequent fluid collection and reactions.

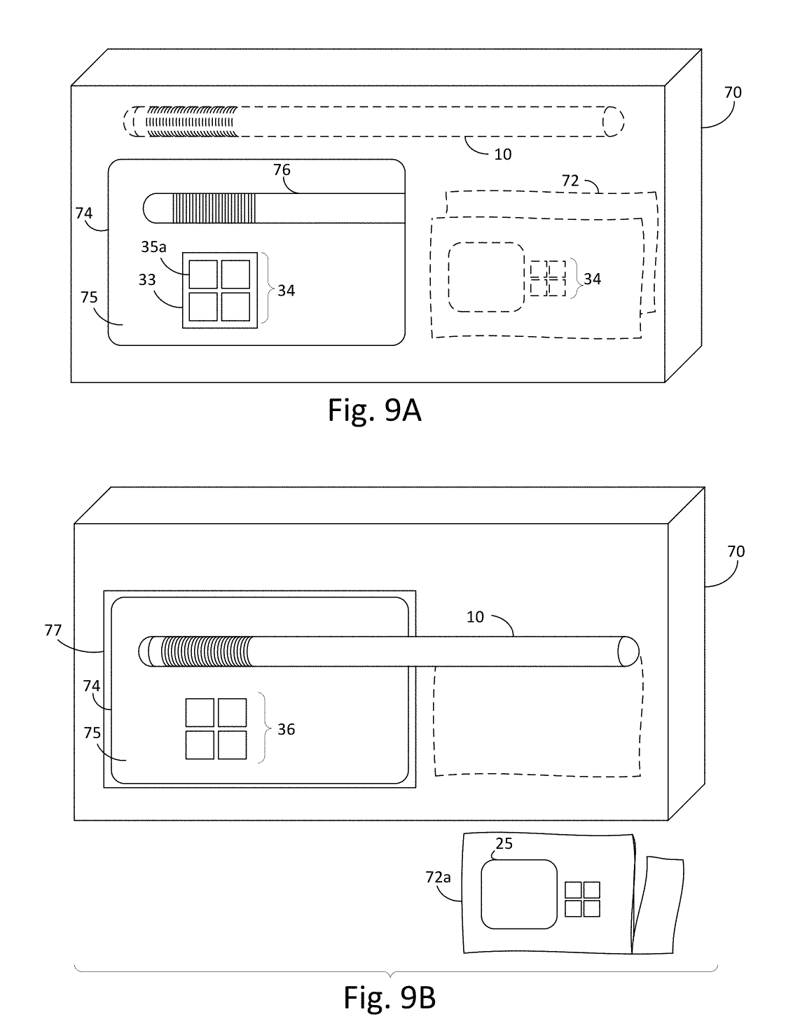

Referring to FIGS. 9A and 9B, the stick 10 is shown received in a container 70. The container can also receive one or more reagent packets 72. The container can include a visual scale area 74 that include a visual scale 36 having a set of colors that are associated with the concentration of glucose levels in the range of 0 to 700 mg/dl or more. In one embodiment, there can be four color area representing 0, 100, 300 and 700 mg/dl. The visual scale area can include a stick placement indicator 76 to assist a user with proper placement of the stick on the visual scale area so that the image of the stick and the set of colors can be captured. The visual scale area can also be included on a card, on the stick or on another medium received in the container. The visual scale area can include background 75 that can have a high contract with the visual scale to improve image capture. The background can be black or nearly black and the visual scale can be hues of green.

The visual color area can include an outer index 77 that can be a contrasting color to the background. The computer readable instructions cause the remote computer device having an image capture system display a corresponding index that can be superimposed on the outer index when viewed with the remote computer device. When the user views the visual scale and post reaction reagent placed on the visual scale area using the remote computer device, the corresponding index can be aligned with the outer index. The remote computer device can automatically capture the image once alignment occurs and the computer readable instructions are provided the location of the hues, visual scale and stick having the post reagent analyte color.

The package can include a reagent window 25 allowing the reagent and post-reaction analyte color to be viewed through the packet. A visual scale 34 can be included on the packet so that the image capture system can capture the post-reaction analyte color and the visual scale simultaneously. The color of the packet can be in high contract to the visual scale.

In operations, the user can open the container and retrieve the stick from within the container. The stick can then be rubbed in a subject's cheek to collet saliva. The stick 10 can then be placed inside a packet 72a of reagent that is retrieved from the container. The stick can then be placed on the visual scale area as indicated. The user can then capture the post-reaction analyte color and the visual scale. The stick can be washed, dried and reused. Additional packets of reagent and the washed and dried stick can be received by the container. In one embodiment, the container can receive one or more packages as shown in FIG. 2.

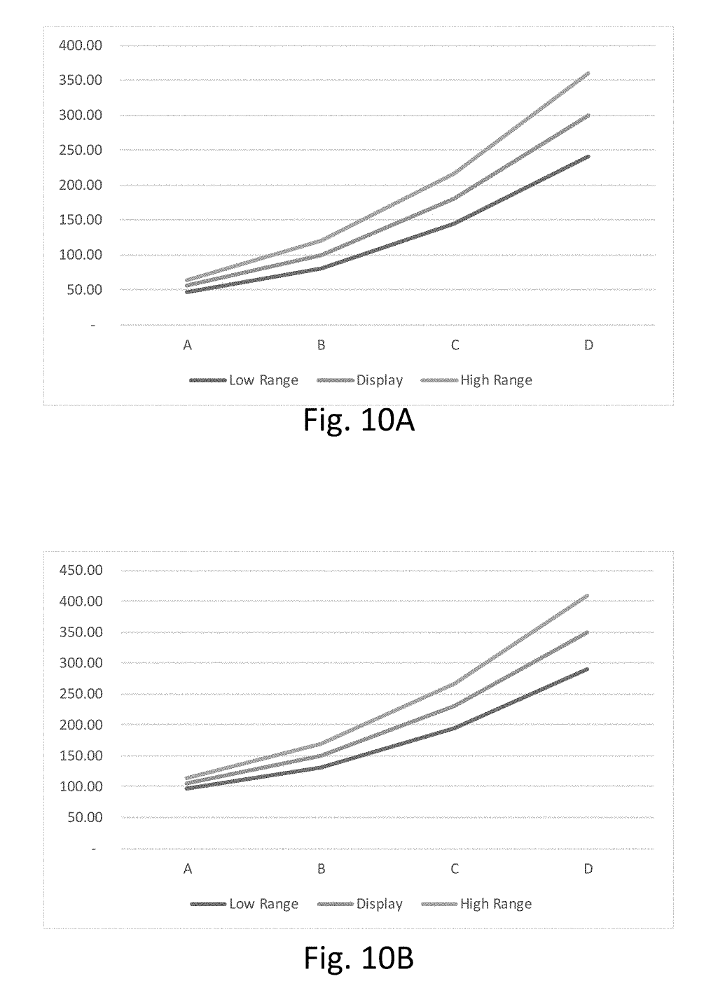

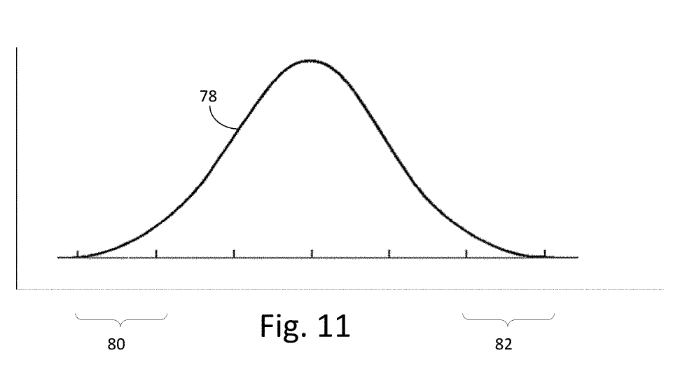

Referring to FIG. 10A, one way in which the computer readable instructions can provide the functionality of the visual scale is represented. Using a four-color scale by way of example, the designation A on the x-axis of the graph represent the color white. B can present the next darkest hue, C the next darkest hue and D the darkest hue. Color A can be the lightest hue in one embodiment. Table 1 illustrates exemplary values for the hues:

TABLE 1

| | Low Range | Display | High Range |
|---|---|---|---|
| Hues | (mg/dl) | | |
| A | 46.75 | 55.00 | 63.25 |
| B | 80.00 | 100.00 | 120.00 |
| C | 144.00 | 180.00 | 216.00 |
| D | 240.00 | 300.00 | 360.00 |

In testing blood glucose, is some embodiment, digital precision of values is unnecessary. The FDA has released its guidance document for self-monitoring blood glucose test systems for use by humans. In its guidance, the FDA states that, for human use, it is sufficient for the device to be proven to have 95% of all results within +/−15% of the comparator results across the entire claimed and measuring range of the device and that 99% of all results are within +/−20% of the comparator results across the entire claimed measuring range of the device. Therefore, with humans, it is sufficient that the displayed reading of the blood glucose monitor fall with in this range. For animals, however, there are no comparable guidelines.

The computer readable instructions can include a lookup table that can include a relationship between visual scale and glucose concentration such that if color A is detected, the glucose concentration can be known to be in the range of 46.75 mg/dl and 63.25 (which is ±15% of 55.00 mg/dl so that 55.00 mg/dl can be displayed to the user and be within 15% of the actual value. In one embodiment, the values that are associated with each color can be modified by the user. For example, different breeds of animals may need different ranges. One study found that smaller breeds had a tendency to have higher blood sugar than medium breeds, and medium breeds had same tendency than the large breeds of dogs. The computer readable instructions can have the ranges vary based upon the user input or the user selecting the breed or other criteria associated with the dog. The same can be provided for other animals such as cats, horses and others. For example, the ranges can be shifted on the y-axis as shown in FIG. 10B. The relationship of the colors to the value range can be linear, quadratic, geometric or other mathematical relationship. The look-up tables and relationship between the color and the values can be extrapolated from testing as well.

The type of test subject can shift the graph associated with the values of the hues. For example, if a first type of test subject has a graph representing the blood glucose values associated with each hue, a second test subject can have these values shifted up, down left, rights or any combination so that the values displayed are matched to the type of test subject. This can be presented as y=f(x) representing the graph of a first type of test subject and y=f(x)±c where c is a shift associated with a second test subject. The first type of test subject y=f(x) can also be shifted by d as shown in the representation y=f(x±d). The combination of shifts can occur as presented by y=f(x±d)±c.

When the remote computer device with the image capture system captures the visual scale and the post reaction analyte, the computer readable instructions can determine the contrast between the plurality of hues of a visual color scale. For example, one hue on the color scale can be assigned the color value A, the second hues assigned a color value B, a third hue assigned a color value C and a fourth hue assigned a color value D. The relationship A→B→C→D can be linear or non-linear. There the numeric difference between A and B and B and C need not be equivalent. The computer readable instructions can use the image of the visual scale and determining a range of color values according to the contrast between each of the hues of the visual color scale and produce a range of values between each hue. The computer readable instructions can assign a color value to the post reaction analyte color and then determine between which hues the post reaction analyte color falls. The computer readable instructions can then determine where in the range of color values between the hues that post analyte reagent color falls and determining a report blood glucose value according to the post reaction analyte color and the range of color values. For example, if A is assigned the numeric value 100, B is assigned the numeric value 200 and the post reagent analyte color is assigned the value 155, the post reaction reagent color fall half way between hues A and B. A can also be assigned a first blood glucose value and B can be assigned a second blood glucose value so that the post reaction reagent color can represent a blood glucose value of half way between the blood glucose values assigned to A and B. The remote computer device an then display the report blood glucose value.

The visual scale area can include a background that has a high contract to the visual scale and/or the colors in the visual scale. The contrast is used by the computer readable instructions to assist with matching the post-reaction analyte color to the visual scale for determining the glucose level to display the user. By using the visual scale and the computer readable instructions, there is no need for calibration of the remote compute device or image capture system as capturing the visual scale with the analyte color allows the hues to be normalized. Further, there is no need to have a controlled ambient light environment since the visual scale and the analyte color and captured in the same lighting environment.

When capturing an image from a tab as in FIGS. 6 and 7, visual scare area of FIGS. 9A and 9B or packet of FIGS. 9A and 9B, the computer readable instructions can determine the greyscale of the image and assign a range of 0 to 100 for black to white to form a curve 78 shown in FIG. 12. From this curve, the low end 80 and the high end 82 of the visual scale can be ignored and image processing preformed on the remaining section. This process can correct for a technological issue that can occur with lighting and the post reaction analyte color. For example, when the subject has high viscosity (e.g. dry) salvia, the stick may need to be agitated vigorously for the reagent and the salvia to property react. This vigorous agitation can result in bubbles or foam developing on or near the collection area or on, in or near the ridges. Bubbles can reflect light from both the inner and outer surface of the bubble. This can result in wavelengths that interfere with each other either destructively or constructively and can results in inaccurate color capture of the images. Therefore, removing the “ends” of the grayscale can reduce these errors. Further, when there are multiple light sources, the process of eliminating the end of the grayscale can reduce errors caused by various light sources and ambient reflections.

In one embodiment, the computer readable instructions can include a calibration function. The computer readable instructions can receive test blood glucose information provided by the user that can results from a lab test, blood test or other external sources that typically high precision in their measurements. These are typically found in medical professional facilities and generally cost prohibitive for wide spread use by consumers. The invention can then be used to determine the report blood glucose value of the test subject. The difference between the test blood glucose information and the report blood glucose value can be compared and the computer readable instructions can use the difference to adjust the reported blood glucose value for the specific test subject.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from the examples and embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A system for determining and reporting results from an analyte taken from a test subject comprising:
a stick having a collection area configured to collect an analyte in a discrete range of volume;
a reagent where the stick with the analyte can be mixed with a reagent to provide a post reaction analyte color;
a visual scale having a plurality of hues and operatively associated with the post reaction analyte color; and,
a set of computer readable instructions included on a remote computer device having an image capture system for: simultaneously capturing a digital image of the visual scale and the post reaction analyte color, determining a contrast between hues of a visual color scale, determining a range of color values according to the contrast between each of the hues of the visual color scale, determining where the post reaction analyte color falls in the range of color values, determining a report blood glucose value according to the post reaction analyte color and the range of color values, and, displaying the report blood glucose value on the remote computer device.

2. The system of claim 1 wherein the analyte is salvia.

3. The system of claim 2 wherein the analyte is canine salvia.

4. The system of claim 1 including a reagent window defined in a package allowing the post reaction analyte color to be viewed which the stick is received in the package.

5. The system of claim 1 wherein the visual scale is disposed on the stick.

6. The system of claim 1 wherein the range of color representing blood glucose values is between 0 mg/dl and 700 mg/dl.

7. The system of claim 1 wherein the collection area includes a pad assembly having a cellulose pad for collecting analyte of a volume wherein a ratio of analyte to reagent is in a range of 1:1 to 1:15.

8. The system of claim 1 wherein the collection area includes a plurality of ridges for collecting analyte of a volume wherein a ratio of analyte to reagent is in a range of 1:1 to 1:15.

9. The system of claim 1 including a visual scale area disposed on an outer surface of a package.

10. The system of claim 1 wherein the reagent is disposed on the collection area.

11. A system for determining and reporting results from an analyte taken from a test subject comprising:
a container having a stick;
a collection area included on the stick configured to collect an analyte;
a reagent received in the container so that when the stick having analyte is mixed with the reagent, a post reaction analyte color is provided;
a visual scale operatively associated with a post-reaction analyte color; and,
a set of computer readable instructions included on a remote computer device having an image capture system for: simultaneously capturing a digital image of the visual scale and the post reaction analyte color, determining a contrast between hues of a visual color scale, determining a range of color values according to the contrast between each of the hues of the visual color scale, determining where the post reaction analyte color falls in the range of color values, determining a report blood glucose value according to the post reaction analyte color and the range of color values, and, displaying the report blood glucose value on the remote computer device.

12. The system of claim 11 wherein the set of computer readable instructions include instructions for transmitting the blood glucose level to a remote computer system.

13. The system of claim 11 wherein the visual scale is disposed on the stick.

14. The system of claim 11 wherein the collection area is configured to collect an analyte in a discrete range of volume.

15. The system of claim 14 wherein the discrete range of volume is in a range of 1 μl to 125 μl.

16. The system of claim 11 wherein. the collection area includes a cellulose pad removably attached to the stick.

17. A system for determining and reporting results from an analyte taken from a test subject comprising:
a stick having a collection area configured to collect an analyte;
a reagent disposed on the stick and mixed with an analyte to provide a post reaction analyte color;
a visual scale operatively associated with the post reaction analyte color; and,
a set of computer readable instructions included on a remote computer device having an image capture system for: simultaneously capturing a digital image of the visual scale and the post reaction analyte color, determining a contrast between hues of a visual color scale, determining a range of color values according to the contrast between each of the hues of the visual color scale, determining where the post reaction analyte color falls in the range of color values, determining a report blood glucose value according to the post reaction analyte color and the range of color values, and, displaying the report blood glucose value on the remote computer device.

18. The system of claim 17 wherein the set of computer readable instructions include instructions for retrieving a set of blood glucose values associated with the hues of a range containing the post reaction analyte color and determining a report blood glucose value from a lookup table and set of blood glucose values.

19. The system of claim 18 wherein the set of computer readable instructions include instructions for modifying the lookup table according to test subject criteria.

20. The system of claim 19 wherein the lookup table includes blood glucose values associated with specific test subject types and the set of computer readable instructions include instructions for selecting a type of test subject where each type of test subject is associated with a different lookup table.
